(19) United States
(12) Patent Application Publication
(10) Pub. No.: US 2003/0050595 A1
(43) Pub. Date: Mar. 13, 2003

(54) Pellet implant system and method of administration

(76) Inventor: Colin B. Campbell, Prescott, AZ (US)

Correspondence Address: PHARMACIA & UPJOHN, 301 HENRIETTA ST, 0228-32-LAW, KALAMAZOO, MI, 49007, US

(21) Appl. No.: 10/235,021

(22) Filed: Sep. 4, 2002

Related U.S. Application Data
(60) Provisional application No. 60/318,228, filed on Sep. 7, 2001.

(51) Int. Cl. A61M 31/00

(52) U.S. Cl. 604/60; 606/116; 606/117; 604/506

(57) ABSTRACT

A pellet implant system and method of implanting a plurality of implants that include at least one pharmaceutical pellet implant and at least one non-pharmaceutical device implant in an animal or human in a single combined procedure. The system includes (1) an implanter apparatus for subcutaneously implanting pharmaceutical pellets or implantable non-pharmaceutical devices in an animal or human through the bore of a hypodermic needle, and (2) a plurality of pellets that include that include at least one pharmaceutical pellet and at least one non-pharmaceutical device pellet sized to be implanted through the needle. The needle is used to puncture the skin of the animal or human. Pellets are delivered under the skin of the animal or human into the through the needle in a single injection. The system minimizes the number of skin punctures required for implanting multiple implants in an animal or human, thus reducing the risk of infection in the animal or human associated with the implant administration.

### CROSS-REFERENCE TO RELATED APPLICATIONS

This application claims the benefit of U.S. provisional application Serial No. 60/318,228 filed on Sep. 7, 2001, under 35 USC 119(e)(i).

### BACKGROUND OF THE INVENTION

1. Field of the Invention

The present invention relates to a pellet implant system and method which administers an implantable non-pharmaceutical pellet implant subcutaneously along with a pharmaceutical pellet implant in a single combined procedure.

2. Description of the Related Art

Implants for delivering doses of pharmaceutical agents to animals and humans are widely used and well known in the art. Classes of pharmaceutical agents for which implants are widely used include, among others, time delayed pharmaceuticals, nutritional supplements, growth stimulants, contraceptive steroids, hormones, antibodies, antigens, biologics, vaccines, prostaglandins, narcotic antagonists, anti-arrhythmics, biocides for flea and parasite control in humans, horses, and domestic animals such as dogs and cats. Implants are commonly used in farm animals in the areas of animal health and production enhancement. For example, growth stimulants administered as implants are commonly used to enhance growth and improve carcass quality of animals which are raised for slaughtering, such as cattle, swine, sheep, turkeys, chickens, and the like. Pharmaceutical implants are generally prepared as small solid pellets and are injected under the skin of the animal.

Implantable identification devices, which can be implanted under the skin or an animal or human used for identification of individual animals or humans, are known in the art. One example of such a device is electronic integrated circuit microchips. The microchip is implanted under the skin of an animal and carries coded information which can be used, for example, to identify an animal when the chip is scanned. Microchips are widely used for the identification of livestock, companion animals, fisheries, and wild animals. The microchips may also contain information such as the animal's medical history, or relevant commercial information. The outbreaks of foot and mouth disease and mad cow disease (bovine spongiform encephalopathy) make identification of carrier animals extremely important. By having an identification device in the animal the veterinarian would know precisely what treatment regimen the animal will have been given.

Microchips are also implanted under the skin of humans. Microchips could contain identifying information which may be useful in detecting abducted children or run-away children. Microchips could also contain vaccination and medical information which could be useful to school personnel. For adults a microchip could contain information that alerts a doctor that the person is epileptic or diabetic.

Microchips and pharmaceutical pellet implants may be administered using similar apparatus and procedure. Generally, pellets or chips are administered by an implanter equipped with a hypodermic needle. The needle is used to puncture the skin of the animal or human. Pellets or chips are forced through the needle and left under the skin as the needle is removed. In the case of farm animals, the pellets or chips are normally implanted under the skin of the ear while an animal is confined in a chute.

Because of the widespread use of implantable identification devices and pharmaceutical implants, it is quite common that one individual animal is implanted with both an identification device implant and a pharmaceutical pellet implant, which is currently done through separate injections. Thus, in such a case, two or more separate punctures have to be made in the skin of an animal.

There are several problems associated with the currently used system and procedures. First, performing the implanting procedure is relative labor-intensive, particularly when a large number of animals are involved. A more troubling problem, which his commonly encountered in administering the implants to a large number of farm animals, is the infection at the puncture site. In the case of farm animals, it is typically that during one implanting session a large number of animals are implanted in rapid sequence, with the same needle often used with as many as 100 or more animals. Moreover, these injections often occur in or near feedlots or other locations with considerably less than ideal sanitary conditions. It is virtually impossible in such situations to provide a sterile injection site on a single animal or to prevent transfer of infective microbes from one animal to the next on the injecting needle. Further complicating the matter is that other procedures may be occurring at the same time as the implanting operation while the animal is confined, such as branding, veterinary inspections or procedures, or the like, which may further excite the animal and make injecting and disinfecting difficult. It is not unusual to even have manure at the injection site. Since these implantations involve the deliberate making of a puncture wound in the animal's skin, bacteria are introduced into the implant sites, either during the delivery of the implant or thereafter, which may cause an infection at the site. Such infections may and often do result in abscesses, which may reduce the effectiveness of the implant by encapsulation of the implant pellet or by pushing the therapeutic pellet out of the original insertion implant receiving puncture, thereby preventing absorption and transport of the active ingredients. It is estimated that from about 10% to about 15% of feedlot cattle which are implanted in the United States subsequently develop abscesses.

The above problems are compounded where an animal is administered both an identification device implant and a pharmaceutical implant through separate skin punctures and separate injections. Multiple injections not only increase the labor requirement for administering the implants, they inevitably put the animal in a higher risk for infections.

A variety of techniques are currently employed to reduce the incidence of infection. Implant manufacturers recommend disinfection of the implanting tool, pellet magazines and needles, and observation of good sanitation practices during the implantation process. The implantation site may be cleaned or disinfected prior to injection to help prevent entry of resident bacteria into the implant receiving puncture and the needle, which may be employed to inject dozens of animals, may be disinfected between animals to prevent the transfer of bacteria from one animal to the next. U.S. Pat. No. 5,817,054 (1998 to Grimm) describes an implanter with a disinfectant dispenser that introduce a disinfectant into the skin puncture wound forming the implant area of each receiving animal. U.S. Pat. No. 5,870,098 (1999 to Stevens and Spurlin) describes an antibiotic and pharmaceutical pellet system and method of providing localized sustained antibiotic release as part of a single therapeutic procedure in order to prevent infection at the injection site. While all such measures may serve to increase sanitation and to reduce initial contamination of the implant receiving puncture, they inevitably increase the cost, time, and labor for administering the implants. Moreover, none of these methods reduce the total number of punctures made in the animal skin, and thus does not reduce the opportunity of entry of microorganisms into the animal body through the punctures. Further, the method described in U.S. Pat. No. 5,817,054 and U.S. Pat. No. 5,870,098 requires the introduction of additional chemical agents, which may have undesirable effects on the animals or on the quality of the animal products.

Accordingly, there is clearly a need for a better system and method of implanting a plurality of pellets including one identification device pellet to an animal.

### SUMMARY OF INVENTION

It is an object of the invention to provide a pellet implant system and method which would reduce the total number of skin punctures required for administering a plurality of implants that include at least one pharmaceutical pellet implant and at least one non-pharmaceutical device implant into an animal or human, thus reducing the opportunity of bacterial entry into the animal through the punctures.

It is another object of the invention to provide a pellet implant system and method which would reduce the risk of infection in an animal caused by the procedure of administering a plurality of implants that include at least one pharmaceutical pellet implant and at least one non-pharmaceutical device implant in an animal or human without introducing additional antibiotic, bacteriostatic, or anti-inflammatory agents into the body of the animal or human.

It is a further object of the invention to provide a pellet implant system and method which would reduce the labor required for administering a plurality of implants that include at least one pharmaceutical pellet implant and at least one non-pharmaceutical device implant in an animal or human.

Other objects of the present invention will become immediately apparent to one skilled in the art upon reading the specification and claims.

These and other objects of the invention are met by providing a pellet implant system and method which permits implanting at least one pharmaceutical pellet implant and at least one non pharmaceutical device implant in an animal in a single injection.

In one aspect, the invention is directed to an implant containing one or more pharmaceutical pellets and one or more non-pharmaceutical device pellets.

In another aspect, the invention is directed to a pellet system and method of implanting a plurality of implants that include at least one pharmaceutical pellet implant and at least one non-pharmaceutical device implant in an animal or human in a single combined procedure. The pellet system includes an implanter apparatus for subcutaneously implanting pharmaceutical pellets or implantable non-pharmaceutical devices in an animal or human through the bore of a hypodermic needle, and a plurality of pellets sized to be implanted through the needle. The pellets include at least one pharmaceutical first pellet and at least one non-pharmaceutical device second pellet, which said pellets are simultaneous delivered as part of a single injection.

In one embodiment the non-pharmaceutical device pellet is an implantable identification device containing information for identifying the animal or human, or source of the animal.

In another embodiment, the non-pharmaceutical device pellet is an implantable device containing information for purposes other than identifying the animal or human, or source of the animal.

In another embodiment the invention provides for a system and method of implanting in an animal or a human a plurality of implants that include at least one non-pharmaceutical device pellet by using an implanter apparatus equipped with a hypodermic needle and an implant magazine remotely coupled to the needle. The implants include at least one pharmaceutical agent pellet and at least one non-pharmaceutical pellet which combined pellets are packaged in the magazine in sequential order for sequential delivery of the pharmaceutical agent and the non-pharmaceutical device.

In a further embodiment, the pharmaceutical pellet contains antibiotics, vaccines, growth promoters, growth stimulants, or anthelmintics, or mixtures thereof.

In still a further embodiment, the implants are implanted in animals selected from cattle, sheep, goat, dog, swine, cat, fish, chickens, turkeys, and humans.

In still a further embodiment, the implants are implanted in animals selected from food animals, companion animals, zoo animals, and exotic animals.

### DEFINITIONS AND CONVENTIONS

The definitions and explanations below are for the terms as used throughout this entire document including both the specification and the claims.

The term “pharmaceutical pellet,” “pharmaceutical implant,” or “pharmaceutical pellet implant” as used herein refers to a physical device (1) that is used to deliver one or more pharmaceutical agents and (2) that is suitable for implantation under the skin of an animal or human.

The term “pharmaceutical agent” as used herein refers to a compound, substance, matter, or composition that is useful for effecting, and is intended to effect, some change in the subject to which it is administered. For example, “pharmaceutical agent” within the scope of this definition includes steroid hormones, prostaglandins, vitamins, antibiotics, antiinflammatory agents, chemotherapeutic agents, cardiovascular and antihypertensive agents. Term “pharmaceutical agent” also includes microorganisms, either living, attenuated or dead, such as bacteria and viruses.

The term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

The term “non-pharmaceutical implant,” “non-pharmaceutical device implant,” or “non-pharmaceutical pellet implant” as used herein refers to any type of physical device, other than pharmaceutical implant as defined above, that is suitable for implantation under the skin of an animal or human. Example of suitable non-pharmaceutical implants for use with the -invention include implantable identification devices for identifying individual subject or source of the subject carrying the implant, or an implantable non-pharmaceutical devices that contains information for purposes other than identifying the animal or humans, such as information about the medical history and relevant commercial information.

The term “implanter” as used herein refers to a device or apparatus used to deliver or insert implants under the skin of an animal or human. Commonly, an implanter has a hypodermic needle through the bore of which the implant is delivered. The term “hypodermic needle” associated with an implanter as used herein refers to a needle, cannula, tubing, or any other structure, flexible or rigid, that has a passageway for receiving and delivering the implant from the implanter into the animal.

### DETAILED DESCRIPTION OF THE INVENTION

All patent applications, patents, and literature references cited in this specification are hereby incorporated by reference in their entirety. In the case of inconsistencies, the present description, including definitions, will control.

The present invention provides for a system and method of administering to an animal or human at least one pharmaceutical implant containing one or more pharmaceutical agents and at least one non-pharmaceutical implant in one combined procedure. Broadly, the implantation system includes (a) an implanter apparatus having a hypodermic needle which is used to implant pharmaceutical agent pellets or implantable microchips through the bore of the hypodermic needle, and (b) at least one pharmaceutical pellet and at least one non-pharmaceutical device pellet. In use, said pharmaceutical pellet and said non-pharmaceutical pellet are loaded into said implanter apparatus and then delivered simultaneously or sequentially to the animal or human in a single injection.

The invention may be practiced on human and any species of animals suitable for receiving implantable non-pharmaceutical implants or pharmaceutical implants, or both as are known in the art. Suitable animals include, but are not limited to, companion animals, food animals, and other domestic animals, wildlife, and zoo animals. Specific examples of suitable animals include, but are not limited to, cattle, horses, goats, pigs, sheep, dog, cat, fish, and exotic species.

The invention may be practiced with any of those implantor apparatus commonly used in the art that are capable of implanting two or more implants in a single injection, such as those described in U.S. Pat. No. 5,522,797, U.S. Pat. No. 5,874,098, and U.S. Pat. No. 4,105,030. The full disclosure of each of U.S. Pat. No. 5,522,797, U.S. Pat. No. 5,874,098, and U.S. Pat. No. 4,105,030 is hereby incorporated herein by reference.

An embodiment of this invention is described below. However, the invention is expressly contemplated to encompass any type of implant system where both a non-pharmaceutical pellet implant and a pharmaceutical pellet implant can be provided to an animal or human. This description is not to be taken in a limiting sense, but is made merely for the purpose of describing the general principles of the invention. The scope of the invention should be determined with reference to the claims.

A suitable implanter apparatus is illustrated and described in detail in U.S. Pat. No. 4,105,030. Generally, the implant apparatus includes a handle, a finger actuatable trigger attached to the handle, a needle, and a rod positioned within the needle for pushing the pellets out of the needle. In use, the needle is loaded with a preferred number of discrete pharmaceutical implants along with at least one non-pharmaceutical implant such as a microchip. An operator grasps the implanter and urges the needle into the hide and under the skin of the target animal to make the implant receiving puncture. Once the needle containing the pellets has been inserted subcutaneously, the operator then depresses the trigger on the handle which causes the needle to be automatically withdrawn by a spring leaving the implanted pellets in place.

Another suitable implanter apparatus is illustrated and described in detail in U.S. Pat. No. 5,522,797, and generally includes a housing having a grip, a trigger attached to the grip, a needle, a pellet magazine strip, a pellet magazine, and an impeller. The pellets to be implanted are loaded into the magazine strip chamber. The needle is used to puncture through the skin or hide of an animal, and the trigger is squeezed to initiate injection of the pellets and so as to cause the impeller to be urged through the magazine chamber and needle bore, thereby forcing the pellets through the bore of needle and into the puncture in the skin. Each magazine strip of the implanter typically contains multiple parallel aligned implants stored in corresponding pellet chambers, which are connected by interconnecting webs. The chambers are arranged in a side-by-side parallel relation. A plurality of strips can be connected in end-to-end relation to increase the implanting capacity before the implanter requires reloading. In use, each magazine chamber is loaded with a preferred number of discrete pharmaceutical pellets along with at least one non-pharmaceutical pellet implant such as a microchip. Thereafter, the magazine is inserted into the implanter housing. An operator grasps the implanter and urges the needle into the hide and under the skin of the target animal to make the implant receiving puncture. The operator then depresses the trigger member activating the propelling mechanism, forcing the implants through the needle bore and into the implant receiving puncture. The operator thereafter withdraws the needle, leaving the implants in the animal.

The non-pharmaceutical pellet implant in the invention is expressly contemplated to encompass any type of physical device, other than pharmaceutical pellet implants, that is suitable for implantation in the body of an animal or human and that can be implanted using the method described therein. Example of suitable non-pharmaceutical pellet implants for use with the invention include, but are not limited to, implantable identification devices for identifying individual animals or humans, and implantable non-pharmaceutical devices that contain information for purposes other than identifying the animal or humans. One example of such implantable identification devices is an implantable electronic transponder or transmitter, commonly called a “microchip.” The microchip contains an unique identification code and is implanted under the skin of a animal or human. These microchips are commercially available from several companies such as Destron Fearing, Trovan, and AVID. Another type of identification device suitable in the invention is illustrated and described in detail in U.S. Pat. No. 4,909,250, which is an implantable identification pellet imprinted with information for identification of an animal.

The implantable non-pharmaceutical, non-identification implant can be a microchip that contains information about the medical history or relevant commercial information.

Any suitable pharmaceutical pellet implant may be used with the present invention. An example of such an implant is one containing one or more pharmaceutical agents in the form of a pellet or a plurality of pellets. The use of the pharmaceutical agent in either liquid or solid forms is specifically contemplated. In the present invention, the nature or type of pharmaceutical agent contained in the pharmaceutical implant is not critical and can be any substance such as enzymes or other organic catalysts, proteins and glycoproteins, peptides, antibodies, nucleic acids, steroids, antibiotics, antimycotics, anti-narcotics, antihistamines, laxatives, vitamins, sedatives, anti-inflammatory substances, antimanics, stimulants, chemotherapeutic agents, contraceptives, radiopharmaceuticals, mineral and nutritional supplements, hormones, pharmaceuticals and other therapeutic agents. The pharmaceutical implants used in the present invention may also be employed for the delivery of microorganisms, either living, attenuated or dead such as bacteria, and viruses such as indigenous vira, enterovira, bacteriophages. The present invention is especially suited for the immediate and sustained delivery of hormones and steroids such as androgens, such as testosterone, trenbolone acetate (TBA), dihydroepiandroterone, and other androgenic steroids, estrogens, such as estradiol-17-β, estradiol benzoate, zeralanone, and other estrogenic steroids, progestins, such as progesterone, melengestrol acetate (MGA), megestrol acetate, medroxyprogesterone acetate, norgestemet, norethidrone, and other progestin compounds, releasing factors, such as leutinizing hormone releasing hormone and analogs, growth hormone releasing hormone and analogs, thyroid releasing hormone and analogs, and other releasing factors and analogs, growth hormones/somatotropin, such as natural and recombinant somatotropins and analogs from various species, growth factors, such as insulin-like growth factor, epidermal growth factor and other such factors. It is also especially suited for delivery of anthelmintics, such as invermectins, and antigens.

Preferred pharmaceutical implants include implants containing pharmaceutical agents for the suppression of estrus and inhibition of pregnancy such as estradiol benzoate. Another preferred pharmaceutical implant is one containing pharmaceutical agents for promoting growth or enhancing body weight gain of animals such as MGA, a combination of MGA and TBA, or a combination of MGA, TBA and estradiol. A number of implants for promoting growth or weight gain in animals are described, for example, in U.S. Pat. Nos. 3, 417,182, 5,091,185, and 5,744,163. Other preferred pharmaceutical agents include antiobiotics such as beta-lactams and cephalosporins. Particularly preferred is the use of ceftiofur in either its sodium salt, hydrochloride salt or free acid form.

It is within the knowledge and skill of those skilled in the art to determine the amount of a pharmaceutical agent used in the implant. See, e.g., U.S. Pat. No. 5,035,891. Generally, the amount of a pharmaceutical agent in the implant will vary depending on the identity of the compound; the size, age, weight, and species of the subject to be treated; the severity of the condition or the magnitude of the effect desired, and so forth. These parameters are easily determined and factored by one of ordinary skill in the art. For example, a representative implant suitable for promoting growth in steers contains a combination of about 200 mg of progesterone and about 20 mg of estradiol benzoate as the pharmaceutical agent. A representative implant suitable for promoting growth in heifers contains a combination of about 200 mg of testosterone propionate and about 20 mg estradiol benzoate as the pharmaceutical agent.

Any of a number of excipients may be employed in the pharmaceutical pellet implant, including polyethylene glycol, magnesium stearate, cellulose and its derivatives, especially ethylcellulose, lactose, polymeric supports and binders and coloring agents. The manufacture of a pharmaceutical pellet implant may be accomplished through a variety of methods known in the art. For example, a process for manufacturing pharmaceutical implants for the delivery of an effective amount of a bioactive peptide or peptide analog over a period of 1 to 12 months is illustrated and described in detail in U.S. Pat. No. 6,159,490, which includes steps of: grinding a copolymer of lactic acid and glycolic acid having a ratio of glycolide to lactide units of from about 0 to 5:1 to a particle size of between about 50 and 150 μm; wetting the ground and copolymer with an aqueous slurry of a bioactive peptide or peptide analog; blending the copolymer and the slurry to obtain a homogeneous mixture of the copolymer and between about 10 and 50% of the bioactive peptide; drying the mixture at reduced pressure and at a temperature not exceeding 25 degree C.; extruding the dried mixture at a temperature between about 70 and 110 degree C.; and cutting cylindrical rods of about 1 to 2 mm diameter and between about 10 and 25 mm in length from the extruded mixture to form the implants.

The descriptions above should be interpreted in the illustrative and not the limited sense. While the invention has been disclosed in connection with the preferred embodiment or embodiments thereof, it should be understood that there may be other embodiments which fall within the scope of the invention as defined by the following claims.

Without further elaboration, it is believed that one skilled in the art can, using the preceding description, practice the present invention to its fullest extent. The following example is provided for purpose of illustrating the invention and is not intended to be limiting upon the scope of the claims. Those skilled in the art will promptly recognize appropriate variations from the procedures both as to reactants and as to reaction conditions and techniques.

### EXAMPLE 1

### Pharmaceutical Pellets Containing Melengestrol Acetate

Pharmaceutical pellets containing melengestrol acetate are formulated by conventional tableting technology, such as wet granulation with water as a granulation liquid or dry granulation, followed by screening, sizing and tablet compression.

| Component | Mg per pellet | |
|---|---|---|
| Melengestrol acetate Micronized | 24 | mg |
| Lactose Monohydrate NF Bolted | 8.235 | mg |
| Sorbitol NF Crystalline | 0.355 | mg |
| Sucrose NF Granular | 0.2755 | |
| Pregelatinized Starch NF | 2.0 | mg |
| Colloidal Silicon Dioxide NF | 0.2 | mg |
| Magnesium Stearate NF Powder Food Grade | 1.0 | mg |

### EXAMPLE 2

### Implantation of an Identification Microchip and Melengestrol Acetate Pellets

Five pharmaceutical pellets of Example 1 and one model TX1400L identification microchip marketed by Electronic ID, Inc. are loaded into the magazine of an implanter apparatus having a hypodermic needle. The operator activates the implanter to first puncture the skin of an animal and then deliver the implant composition through the needle and into the animal. In the case where the animal is a heifer, it is preferred that the puncture occurs at the posterior portion of the ear. The pharmaceutical pellets of the implant deliver the melengestrol acetate in an amount and rate sufficient to promote growth, suppress estrus, and inhibit pregnancy, while the microchip provides information identifying the animal when the microchip is scanned with a HS59001-F Mini Portable Reader marketed by Electronic ID, Inc.

1. A method for implanting at least one pharmaceutical pellet and at least one non-pharmaceutical pellet under the skin of an animal or human in a single injection, comprising the steps of:
a) providing an implanter apparatus for implanting pellets in an animal through the bore of a hypodermic needle;
b) loading said implanter apparatus with at least one pharmaceutical pellet and at least one non-pharmaceutical pellet; and
c) inserting the hypodermic needle under the skin of the animal and injecting said pharmaceutical pellet and said non-pharmaceutical pellet under the skin of said animal.

2. The method according to claim 1 wherein said implanter apparatus is equipped with an pellet magazine and wherein the said magazine is loaded with said pharmaceutical pellet and said non-pharmaceutical pellet.

3. The method according to claim 1 including the steps of:
(a) inserting the hypodermic needle under the skin of the animal or human and injecting said pharmaceutical pellet, and while
(b) maintaining the hypodermic needle in place under the skin of the animal or human, sequentially injecting said non-pharmaceutical pellet.

4. The method according to claim 1 including the steps of:
(a) inserting the hypodermic needle under the skin of the animal or human and injecting said non-pharmaceutical pellet, and while
(b) maintaining the hypodermic needle in place under the skin of the animal or human, sequentially injecting said pharmaceutical pellet.

5. The method of according to claim 1 wherein said non-pharmaceutical pellet is an identification device.

6. The method according to claim 5 wherein said identification device is a microchip.

7. The method according to claim 5 wherein said identification device is a transponder or transmitter.

8. The method according to claim 1 wherein said pharmaceutical pellet contains one or more pharmaceutical agents that comprise antibiotics, vaccines, growth promoters, growth stimulants, anthelmintics and mixtures thereof.

9. The method according to claim 8 wherein said pharmaceutical agent comprises MGA, a combination of MGA and TBA, or a combination of MGA, TBA and estradiol.

10. The method according to claim 1 including the step of providing a plurality of discrete pharmaceutical pellets.

11. The method according to claim 1 wherein said animal is selected from the group consisting of cattle, sheep, goat, dog, swine, cat, fish, chicken, and turkey.

12. The method according to claim 1 wherein said animal is selected from the group consisting of food animals, companion animals, exotic animals, zoo animals, and exotic animals.

13. An implant for subcutaneous implantation in an animal or human comprising:
(a) at least one non-pharmaceutical pellet; and
(b) at least one pharmaceutical pellet.

14. The implant according to claim 12 wherein said non-pharmaceutical pellet is an identification device.

15. The implant according to claim 13 wherein said identification device is microchip.

16. The implant according to claim 13 wherein said identification device is a transponder or transmitter.

17. The implant according to claim 13 wherein said pharmaceutical pellet contains one or more pharmaceutical agents that comprise antibiotics, vaccines, growth promoters, growth stimulants, anthelmintics and mixtures thereof.

18. The method according to claim 17 wherein said pharmaceutical agent comprises MGA, a combination of MGA and TBA, or a combination of MGA, TBA and estradiol.

19. The implant according to claim 13 wherein all said pellets are joined in a single unit.

20. A pellet implant system for implanting at least one pharmaceutical pellet and at least one non-pharmaceutical device pellet under the skin of an animal in one combined procedure, comprising
(a) an implanter apparatus for implanting pellets in an animal through the bore of a hypodermic needle; and
(b) at least one pharmaceutical pellet and at least one non-pharmaceutical device pellet, wherein all said pellets are loaded into said implanter apparatus for simultaneous delivery to the animal in a single injection.

21. The pellet implant system according to claim 18 wherein said implanter apparatus is equipped with an pellet magazine and wherein the said magazine is loaded with said pharmaceutical pellet and said non-pharmaceutical pellet.

22. The pellet implant system according to claim 18 wherein said non-pharmaceutical pellet is an identification device.

23. The pellet implant system according to claim 18 wherein said identification device is a microchip.

24. The pellet implant system according to claim 18 wherein said identification device is a transponder or transmitter.

25. The pellet implant system according to claim 18 wherein said pharmaceutical pellet contains one or more pharmaceutical agents that comprise antibiotics, vaccines, growth promoters, growth stimulants, anthelmintics and mixtures thereof.

26. The method according to claim 25 wherein said pharmaceutical agent comprises MGA, a combination of MGA and TBA, or a combination of MGA, TBA and estradiol.
